(19) United States
(12) Patent Application Publication
(10) Pub. No.: US 2025/0275775 A1
(43) Pub. Date: Sep. 4, 2025

(54) GUIDANCE TOOLS, SYSTEMS, AND METHODS

(71) Applicant: Wright Medical Technology, Inc., Memphis, TN (US)

(72) Inventor: Meghan Kubacki, Memphis, TN (US)

(73) Assignee: Wright Medical Technology, Inc., Memphis, TN (US)

(21) Appl. No.: 19/212,868

(22) Filed: May 20, 2025

Related U.S. Application Data
(62) Division of application No. 17/776,893, filed on May 13, 2022.
(60) Provisional application No. 62/962,610, filed on Jan. 17, 2020.

(51) Int. Cl. A61B 17/17; A61B 17/00; A61B 17/16

(52) U.S. Cl. CPC A61B 17/1717; A61B 17/164; A61B 2017/00867

(57) ABSTRACT

A guidance tool includes a body having a length extending from a first end to a second end. The body includes a shape memory section along the length of the body. The shape memory section has a curved shape.

### CROSS-REFERENCE TO RELATED APPLICATIONS AND INCORPORATION BY REFERENCE

This application is a divisional filed under 37 C.F.R. § 1.53 claiming the benefit under 35 U.S.C. § 120 of any and all applications for which a foreign or domestic priority claim is identified in the Application Data Sheet as filed with the present application, including U.S. patent application Ser. No. 17/776,893, filed May 13, 2022, International Patent Application No. PCT/US2020/065189, filed Dec. 16, 2020, and U.S. Provisional Patent Application No. 62/962,610, filed Jan. 17, 2020, and are hereby incorporated by reference in accordance with 37 C.F.R. §§ 1.57; 1.97; and 1.98 in their entireties This application also incorporates by reference the entire disclosures of commonly assigned U.S. Pat. No. 8,808,303, entitled “Orthopedic Surgical Guide;” U.S. Pat. No. 9,675,365, entitled “System and Method for Anterior Approach for Installing Tibial Stem;” and U.S. Pat. No. 10,456,179, entitled “Intramedullary Ankle Technique and System.”.

### FIELD OF DISCLOSURE

The disclosed systems and methods relate to surgical tools and methods. More particularly, the disclosed systems and methods relate to guiding a flexible reamer, including flexible reamer that may be used to form an intramedullary canal.

### BACKGROUND

Many surgical procedures use rotating cutting tools, such as reamers, to form cavities or channels within bone. One example of such a surgical procedure is a total ankle replacement (“TAR”) procedure in which an intramedullary channel may be formed in a tibia so that the tibia may receive a stem component. The intramedullary channel typically is formed along the mechanical axis of the tibia, and many conventional techniques require the violation of additional bones beyond the tibia (e.g., the talus and calcaneus) to form the intramedullary channel. One example of such a technique is disclosed in commonly assigned U.S. Pat. No. 8,808,303, which has been incorporated by reference above. Violating additional bones beyond the tibia may increase the length of the surgery and risk of infection or other complications.

### SUMMARY

In some embodiments, a guidance tool includes a body having a length extending from a first end to a second end. The body includes a shape memory section along the length of the body. The shape memory section has a curved shape.

In some embodiments, a method includes inserting a first end of a guidance tool into an end of a bone; advancing the guidance tool into the bone until a shape memory section of the guidance tool is disposed adjacent to the end of the bone; cutting the guidance tool at a location between the shape memory section and a second end of the guidance tool; and advancing a cutting tool along the guidance tool to form a cavity in the bone. Cutting the guidance tool allows the shape memory section of the guidance tool to regain its programmed shape.

In some embodiments, a method includes coupling a fixture along a length of a bone such that a hole defined by the fixture is positioned adjacent to a side of the bone; inserting a guidance tool through the hole of the fixture and into the bone until the guidance tool extends along a length of the bone and a leading end of the guidance tool is exposed adjacent to an end of the bone; and advancing a cutting tool along the guidance tool to form a cavity in the bone.

### BRIEF DESCRIPTION OF THE DRAWINGS

FIG. 1 illustrates one example of a human foot;

FIG. 2 illustrates one example of a resected tibia in accordance with some embodiments;

FIG. 3 illustrates one example of a resected tibia and a resected talus in accordance with some embodiments;

FIG. 4 is a frontal plane view of one example of a guidance tool being inserted into a tibia in accordance with some embodiments;

FIG. 5 is a sagittal plane view of a cutting tool being guided by the guidance tool shown in FIG. 4 in accordance with some embodiments;

FIG. 6 is a frontal plane view of one example of a fixture and guidance tool in accordance with some embodiments; and

FIG. 7 is a sagittal plane view of the fixture illustrated in FIG. 6 in accordance with some embodiments.

### DETAILED DESCRIPTION

This description of preferred embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

The disclosed systems and methods advantageously facilitate the intramedullary guidance while minimizing and/or eliminating the violation of adjacent bones as is typically done during conventional surgical procedures. While the systems and methods are described in connection with performing TAR, one of ordinary skill in the art will understand that the disclosed systems and methods may be used to facilitate the creation of intramedullary canals or channels in other bones or body parts.

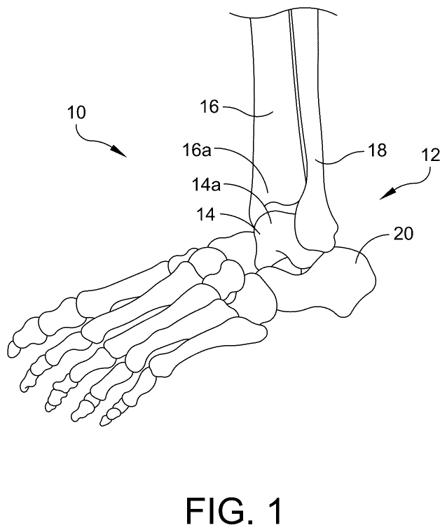

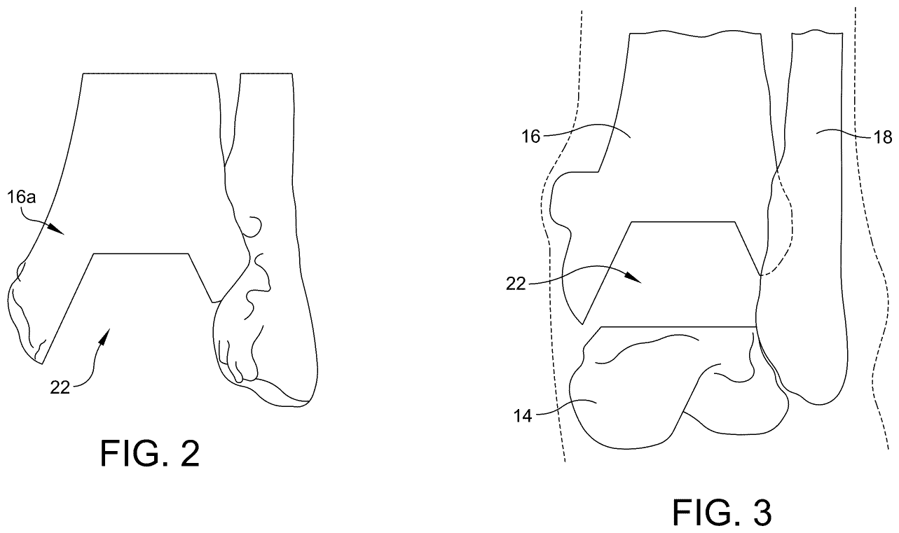

FIG. 1 illustrates one example of a human foot 10 and ankle 12. As is known, the human foot 10 includes a number of bones, including the talus 14, which sits atop the calcaneus 20. The talus 14 forms part of the ankle joint with the tibia 16, which is positioned adjacent to the fibula 18. To prepare an ankle 12 for a TAR, the talus 14 and tibia 16 may be resected to provide a resected joint space 22 as best seen in FIGS. 2 and 3. Examples of tools and procedures for forming the resected joint space 22 are disclosed in U.S. Pat. Nos. 8,808,303 and 9,675,365, which have been incorporated by reference above.

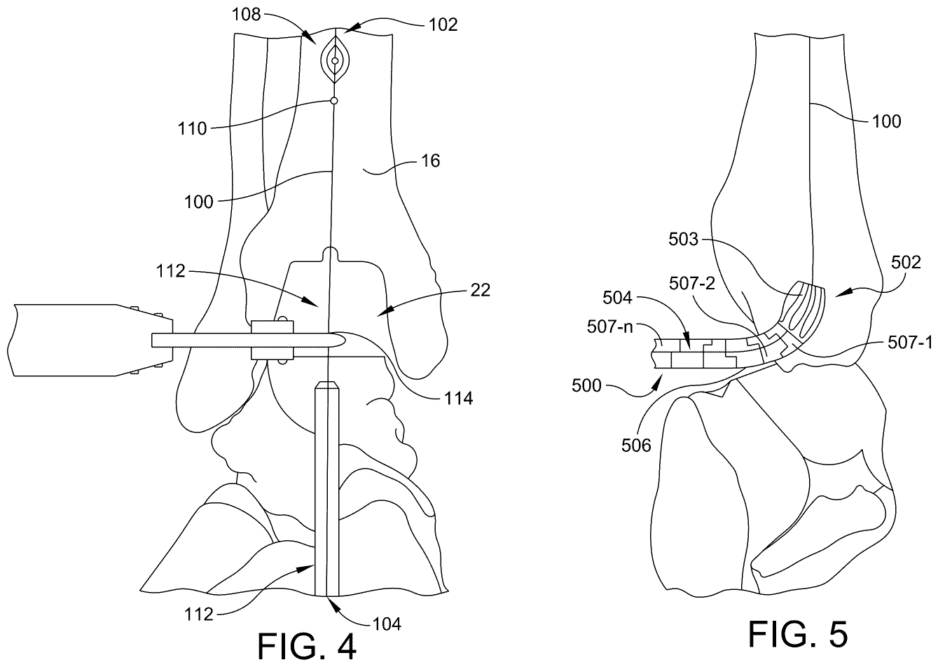

FIGS. 4 and 5 illustrate one example of a guidance tool in accordance with some embodiments. Guidance tool 100 has an elongate shape extending from a first end 102, which may be an insertion end, to a second end 104, which may be a trailing end. End 102 may include a point or taper for facilitating insertion of the guidance tool 100 into a medium, such as skin or bone. Guidance tool 100 may be formed from shape memory material, such as a shape memory alloy selected from the group consisting of Cu—Al—Ni, NiTi (e.g., Nitinol), Fe—Mn—Si, Cu—Zn—Al, and Cu—Al—Ni.

In some embodiments, guidance tool 100 may include a centering mechanism 108 for centering the guidance tool within a bone. For example, the centering mechanism 108 may include a balloon or stent material configured to expand from a first, collapsed configuration to a second, expanded configuration. The guidance tool 100 is inserted into the medium (e.g., an intramedullary space, such as cancellous bone) with the centering mechanism 108 in its collapsed configuration. When guidance tool 100 has been inserted to its desired depth or location, the centering mechanism 108 may be deployed into its expanded configuration.

As will be understood by one of ordinary skill in the art, the manner in which the centering mechanism 108 is deployed may vary. For example, in embodiments where the centering mechanism 108 includes a balloon, then a gas, gel, or liquid may be injected into the internal chamber defined by the balloon to increase the size of the balloon. Techniques similar to those used in vertebroplasty and kyphoplasty may be used to expand the balloon. One of ordinary skill in the art will understand that as the diameter of the balloon increases the cancellous bone is compressed against the stronger cortical bone thereby centering the guidance tool within the bone. Additional materials, such as a stent material, may be provided along with a balloon material to guide the expansion and shape of the balloon.

In some embodiments, guidance tool 100 includes a stop 110 along its length. For example, stop 110 may take the form of a bead, taper, or other protrusion having an enlarged diameter relative to one or more portions of guidance tool 100 that are adjacent to stop 110. Stop 110 may be disposed adjacent to centering mechanism 108 and is configured to stop the advancement of a cutting tool along the guidance tool 100, as will be described in greater detail below, thereby protecting the centering mechanism 108 from being damaged by the cutting tool.

Guidance tool 100 may include a shape memory section or portion 112. In some embodiments, the shape memory section 112 is curved to facilitate the guidance of a flexible cutting tool into an intramedullary canal. As described in greater detail below, the shape memory section 112 may be positioned along the length of the guidance tool 100 such that when the stop 110 is located at the desired location within the bone the shape memory section will be adjacent to an end of the bone.

In some embodiments, the guidance tool 100 may be placed within an inferior portion of a tibia 16 with the aid of a cannula. The tibia 16 and talus 14 may be prepared by making bony cuts, such as those described in U.S. Pat. No. 9,675,365, entitled “System and Method for Anterior Approach for Installing Tibial Stem,” which is incorporated by reference herein in its entirety. Once the inferior portion of the tibia 16 is resected to form a resected joint space 22, the cannula may be positioned adjacent to the calcaneus 20.

With the cannula positioned, the leading end 102 of guidance tool 100 is inserted into the cannula and advanced into the calcaneus 20, talus 14, and tibia 16. The position of the guidance tool 100 within the tibia may be checked using fluoroscopy as will be understood by one of ordinary skill in the art. In some embodiments, the guidance tool 100 may include one or more markings (not shown) along its length. Each marking may identify a distance from the respective marking to the leading end 102 of the guidance tool 100 or a distance from the respective marking to the stop 110 such that a surgeon or physician will be able to determine if the guidance tool 100 has been inserted to the desired depth without the use of fluoroscopy.

In embodiments in which the guidance tool 100 is configured with a centering mechanism 108, the centering mechanism may be activated or deployed to center the guidance tool 100 within the tibia 16. For example, if the centering mechanism 108 includes a balloon that may be inflated around the guidance tool 100, then the balloon is inflated, such as by injecting a gas, gel, or liquid into the internal chamber defined by the balloon. As the balloon is filled, the balloon expands, which results in the cancellous being compressed. The cancellous bone is compressed to the stronger cortical bone which is sufficiently strong to withstand the pressure exerted by the balloon being inflated.

With the guidance tool 100 positioned within the tibia 16, the shape memory section 112 may be deployed. In some embodiments, the shape memory section 112 is deployed by cutting the guidance tool 100, such as by using a pin cutter, adjacent to the shape memory section 112 to form a cut end 114. The cutting of the guidance tool 100 eliminates the straightening force that was being applied to the guidance tool 100 by the cannula such that the shape memory section 112 is now free to revert back to its programmed shape, which may be a curved shape. In some embodiments, the curved shape of the shape memory section 112 extends through the anterior window of the resected joint space 22. In some embodiments, the shape memory section 112 may be deployed prior to positioning the cannula and activating or deploying the centering mechanism as will be understood by one of ordinary skill in the art.

The guidance tool 100 is now ready to guide a cutting tool, such as a flexible reamer 500, to prepare an intramedullary channel for receiving an implant. In some embodiments, a flexible reamer, such as the flexible reamer 500 described in U.S. Pat. No. 10,456,179, entitled “Intramedullary Ankle Technique and System,” the entirety of which is incorporated by reference herein, is modified to provide a cannulated flexible reamer. The cannulated flexible reamer 500 defines a passageway 504 that extends through the nose 502 and the rest of the reamer 500. In some embodiments, the body 506 of the cannulated flexible reamer 500 includes a plurality of segments. The segments 507-1, 507-2, . . . 507-n (collectively, “segments 507”) may be movable (e.g., rotatable and/or pivotable) relative to an adjacent segment 507. In some embodiment, each segment 507 is movable (e.g., rotatable and/or pivotable) relative to an adjacent segment such that the reamer 500 may bend along its length.

The cannulated flexible reamer 500 is inserted over the guidance tool 100. More particularly, the leading end or nose 502 of the cannulated flexible reamer 500, which may include one or more flutes or cutting surfaces 503, is slid onto the cut end 114 of guidance tool 100 such that the guidance tool 100 is received within passageway 504 of reamer 500 as shown in FIG. 5. The flexible reamer 500 is advanced along the guidance tool 100, including along the shape memory section 112, and up into the tibia 16 to form an intramedullary channel. The flexible reamer is advanced along the length of the guidance tool 100 until it contacts stop 110 or till a desired depth has been achieved. In some embodiments, the guidance tool 100 may include markings along its length to provide a visual aid to the surgeon.

Once the intramedullary channel has been formed, the cannulated flexible reamer 500 may be slid off the guidance tool 100. The guidance tool 100 may then be removed from the tibia 16. In some embodiments in which the guidance tool 100 includes a centering mechanism 108 in the form of a balloon, the balloon may be deflated prior to removing the guidance tool 100 from its placement within the tibia 16.

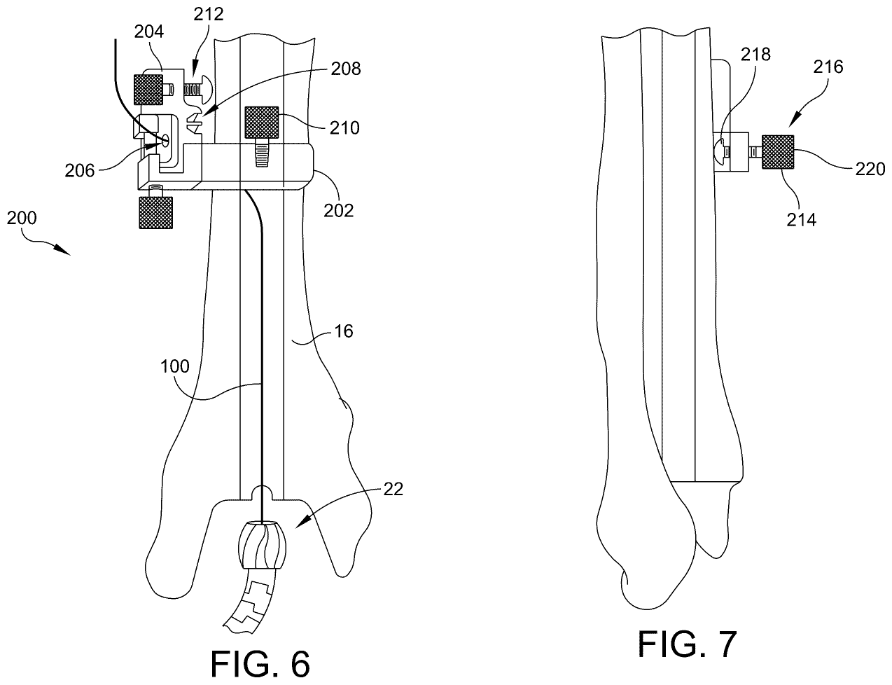

FIGS. 6 and 7 illustrate another example of the insertion of a guidance tool into a bone canal in accordance with some embodiments. As shown in FIGS. 6 and 7, the guidance tool 100 is inserted into the bone canal with the assistance of a fixture 200. Fixture 200 may include a body 202 that may be coupled to the bone or to an extramedullary guidance device, such as, for example, a foot holder and alignment tool 300 described in U.S. Pat. No. 8,808,303, as will be understood by one of ordinary skill in the art.

In some embodiments, body 202 is sized and configured such that the length of the body 202 extends across or substantially across a width of a bone, such as a human tibia 16. Body 202 of fixture 200 may include a flange 204 extending at an angle (e.g., perpendicularly) with respect to a longitudinal axis defined by the body 202. Body 202 defines at least one hole 206 sized and configured to receive a guidance tool, such as guidance tool 100 described above or a conventional k-wire. In some embodiments, hole 206 is configured to allow the guidance tool to be inserted at a non-orthogonal angle relative to an axis defined by the bone (e.g., a mechanical axis or a longitudinal axis). Body 202 may also include one or more gunsights 208 for providing an alignment check.

Body 202 may also include one or more adjustment mechanisms, including an anterior-posterior (“AP”) adjustment block 210, a medial-lateral (“ML”) adjustment block 212, and a proximal-distal adjustment block 214. The implementation of the adjustment mechanisms may be varied. For example, in some embodiments, the adjustment mechanisms include a threaded thumb screw 216 having an enlarged foot 218 that is disposed at an opposite end of thumb wheel 220. In some embodiments, the foot 218 is sized and configured to contact, but not cause damage or dig into, a bone, body 202 of fixture 200, or the extramedullary guidance tool 100 as will be understood by one of ordinary skill in the art.

In use, the fixture 200 is positioned along a length of a bone, such as a tibia 16, after the bone has been prepared to accept a guidance tool 100. For example, in embodiments in which the fixture 200 and guidance tool 100 are to be used to guide a flexible reamer, e.g., flexible reamer 500, to form an intramedullary channel for receiving a tibial component of an ankle prosthesis, the inferior portion 16a of the tibia 16 is resected to form a resected joint space 22 as shown in FIGS. 2 and 3. The resected joint space may be formed as described in U.S. Pat. No. 9,675,365, entitled “System and Method for Anterior Approach for Installing Tibial Stem,” which has been incorporated by reference above.

The fixture 200 may be positioned along the length of the bone by using a strap (not shown) to couple the fixture 200 directly to the bone or by coupling the fixture 200 to an external guidance device (also not shown). Positioning of the fixture 200 along the length of the bone may also include adjusting the location of the fixture 200 using one or more of the adjustment mechanisms, i.e., AP adjustment block 210, ML adjustment block 212, and proximal-distal adjustment block 214. The adjustment mechanisms may be used by rotating the respective threaded thumb screw 216 as will be understood by one of ordinary skill in the art. The alignment of the fixture 200 relative to the bone may be checked using gunsights 208, which may include visualizing the fixture and bone using fluoroscopy as will be understood by one of ordinary skill in the art.

Once the fixture 200 is positioned, a guidance tool 100 may be inserted into the hole 206 of fixture 200. Hole 206 may be positioned along a medial or lateral side of the bone such that the guidance tool 100 is inserted into a medial or lateral aspect of the bone. The guidance tool 100 is routed through fixture 200 and along the length of the bone until a leading end extends from the inferior portion of the tibia 16 into resected joint space 22. For example, in some embodiments, the guidance tool 100 is pre-bent or has sufficient flexibility such that when the guidance tool 100 is inserted through the fixture at an angle it may be manipulated by the surgeon until the desired orientation within the bone is achieved.

With the leading end of the guidance tool 100 exposed within the resected joint space 22, a cutting tool, such as a flexible reamer, is used to prepare an intramedullary channel for receiving an implant. As described above, a flexible reamer, such as the flexible reamer described in U.S. Pat. No. 10,456,179, entitled “Intramedullary Ankle Technique and System” and which has been incorporated by reference above, is modified to provide a cannulated flexible reamer that is inserted over the leading end of guidance tool. The flexible reamer is advanced along the guidance tool 100 and up into the tibia 16 to form an intramedullary channel.

Once the intramedullary channel has been formed, the cannulated flexible reamer may be slid off of the guidance tool 100. The guidance tool 100 may then be removed from the tibia 16. Fixture 200 may then also be removed from its engagement with the bone.

In some embodiments, a guidance tool includes a body having a length extending from a first end to a second end. The body includes a shape memory section along the length of the body. The shape memory section has a curved shape.

In some embodiments, a stop is positioned along the length of the body adjacent to the first end. The stop has an enlarged dimension relative to portions of the body that are directly adjacent to the stop.

In some embodiments, the body includes a centering mechanism disposed between the stop and the first end of the body.

In some embodiments, the centering mechanism includes an expandable portion configured to expand from a collapsed configuration to an expanded configuration.

In some embodiments, the centering mechanism includes a balloon.

In some embodiments, the shape memory section is positioned between the stop and the second end of the body.

In some embodiments, the body is formed from a shape memory alloy.

In some embodiments, the shape memory alloy is selected from the group consisting of Cu—Al—Ni, NiTi, Fe—Mn—Si, Cu—Zn—Al, and Cu—Al—Ni.

In some embodiments, a method includes inserting a first end of a guidance tool into an end of a bone; advancing the guidance tool into the bone until a shape memory section of the guidance tool is disposed adjacent to the end of the bone; cutting the guidance tool at a location between the shape memory section and a second end of the guidance tool; and advancing a cutting tool along the guidance tool to form a cavity in the bone. Cutting the guidance tool allows the shape memory section of the guidance tool to regain its programmed shape.

In some embodiments, the method includes expanding a centering mechanism of the guidance tool to center the guidance tool within the bone prior to cutting the guidance tool.

In some embodiments, expanding the centering mechanism includes inflating a balloon.

In some embodiments, the cutting tool includes a cannulated flexible reamer.

In some embodiments, the cutting tool is advanced along the guidance tool until the cutting tool contacts a stop disposed adjacent to a first end of the guidance tool.

In some embodiments, the method includes resecting an end of the bone to form a resected joint space between the bone and a second bone prior to inserting the end of the guidance tool into the end of the bone.

In some embodiments, a cut end of the guidance tool extends through a window of the resected joint space when the shape memory section of the guidance tool regains its programmed shape.

In some embodiments, the bone is a tibia.

In some embodiments, a method includes coupling a fixture along a length of a bone such that a hole defined by the fixture is positioned adjacent to a side of the bone; inserting a guidance tool through the hole of the fixture and into the bone until the guidance tool extends along a length of the bone and a leading end of the guidance tool is exposed adjacent to an end of the bone; and advancing a cutting tool along the guidance tool to form a cavity in the bone.

In some embodiments, the method includes forming a resected joint space between the bone and a second bone prior to inserting the guidance tool into the bone.

In some embodiments, the bone is a tibia.

In some embodiments, the cutting tool is a cannulated flexible reamer.

In some embodiments a system includes a guidance tool and a cannulated flexible reamer. The guidance tool has a body with a length extending from a first end to a second end. The body includes a shape memory section along the length of the body. The shape memory section has a curved shape. The cannulated flexible reamer defining a channel sized and configured to receive at least a portion of the guidance tool. A leading end of the cannulated flexible reamer includes plurality of flutes.

In some embodiments, a body of the cannulated flexible reamer includes a plurality of segments. At least one of the segments is movable relative to an adjacent segment. In some embodiments, each segment is movable relative to an adjacent segment.

In some embodiments a system includes a guidance tool, a cannulated flexible reamer, and a fixture. The guidance tool has a body with a length extending from a first end to a second end. The body includes a shape memory section along the length of the body. The shape memory section has a curved shape. The cannulated flexible reamer defining a channel sized and configured to receive at least a portion of the guidance tool. A leading end of the cannulated flexible reamer includes plurality of flutes. The fixture is sized and configured to be disposed along a length of a bone. The fixture defines a hole that is positioned along the length of fixture such that, when the fixture is disposed adjacent to a bone, the guidance tool may be received in the hole of the fixture and into the bone.

In some embodiments, a body of the cannulated flexible reamer includes a plurality of segments. At least one of the segments is movable relative to an adjacent segment. In some embodiments, each segment is movable relative to an adjacent segment.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. A method, comprising:
inserting a first end of a guidance tool into an end of a bone;
advancing the guidance tool into the bone until a shape memory section of the guidance tool is disposed adjacent to the end of the bone;
cutting the guidance tool at a location between the shape memory section and a second end of the guidance tool, wherein cutting the guidance tool causes the shape memory section of the guidance tool to regain its programmed shape; and
advancing a cutting tool along the guidance tool to form a cavity in the bone.

2. The method of claim 1, further comprising an expanding centering mechanism of the guidance tool to center the guidance tool within the bone.

3. The method of claim 2, wherein expanding the centering mechanism includes inflating a balloon.

4. The method of claim 1, wherein the cutting tool includes a cannulated flexible reamer.

5. The method of claim 1, wherein the cutting tool is advanced along the guidance tool until the cutting tool contacts a stop disposed adjacent to a first end of the guidance tool.

6. The method of claim 1, further comprising resecting an end of the bone to form a resected joint space between the bone and a second bone prior to inserting the end of the guidance tool into the end of the bone.

7. The method of claim 6, wherein a cut end of the guidance tool extends through a window of the resected joint space when the shape memory section of the guidance tool regains its programmed shape.

8. The method of claim 1, wherein the bone is a tibia.

9. A method comprising:
coupling a fixture along a length of a bone such that a hole defined by the fixture is positioned adjacent to a side of the bone;
inserting a guidance tool through the hole of the fixture and into the bone until the guidance tool extends along a length of the bone and a leading end of the guidance tool is exposed adjacent to an end of the bone; and
advancing a cutting tool along the guidance tool to form a cavity in the bone.

10. The method of claim 9, further comprising forming a resected joint space between the bone and a second bone prior to inserting the guidance tool into the bone.

11. The method of claim 10, wherein the bone is a tibia.

12. The method of claim 9, wherein the cutting tool is a cannulated flexible reamer.

13. A method comprising:
coupling a fixture along a length of a bone such that a hole defined by the fixture is positioned adjacent to a side of the bone;
providing a guidance tool, including a body having a length extending from a first end to a second end, the body including a shape memory section along the length of the body, the shape memory section having a curved shape, a centering mechanism disposed between the stop and the first end of the body, an expandable portion configured to expand from a collapsed configuration to an expanded configuration, a stop is positioned along the length of the body adjacent to the first end, the stop having an enlarged dimension relative to portions of the body that are directly adjacent to the stop, wherein the shape memory section is positioned between the stop and the second end of the body;
inserting a guidance tool through the hole of the fixture and into the bone until the guidance tool extends along a length of the bone and a leading end of the guidance tool is exposed adjacent to an end of the bone; and
advancing a cutting tool along the guidance tool to form a cavity in the bone.

14. The method of claim 13, expanding the centering mechanism of the guidance tool to center the guidance tool within the bone.

15. The method of claim 14, wherein expanding the centering mechanism includes inflating a balloon.

16. The method of claim 13, wherein the cutting tool includes a cannulated flexible reamer.

17. The method of claim 13, wherein the cutting tool is advanced along the guidance tool until the cutting tool contacts the stop disposed adjacent to the first end of the guidance tool.

18. The method of claim 13, further comprising resecting an end of the bone to form a resected joint space between the bone and a second bone prior to inserting the end of the guidance tool adjacent to the end of the bone.

19. The method of claim 18, wherein a cut end of the guidance tool extends through a window of the resected joint space when the shape memory section of the guidance tool regains its programmed shape.

20. The method of claim 13, wherein the bone is a tibia.
